(19) United States
(12) Patent Application Publication
(10) Pub. No.: US 2024/0398332 A1
(43) Pub. Date: Dec. 5, 2024

(54) DENTAL IMPRESSION ACQUISITION KIT AND METHODS OF USER THEROF

(71) Applicant: Dorothy Kassab, Fayetteville, NY (US)

(72) Inventor: Dorothy Kassab, Fayetteville, NY (US)

(21) Appl. No.: 18/678,477

(22) Filed: May 30, 2024

Related U.S. Application Data
(60) Provisional application No. 63/504,849, filed on May 30, 2023.

(51) Int. Cl. A61B 5/00; A61B 90/00; A63B 71/08

(52) U.S. Cl. CPC A61B 5/4547; A61B 90/06; A63B 71/085; A61B 2090/061

(57) ABSTRACT

The present technology relates to a kit for acquisition of human anatomy and a relationship of structures to each other for therapeutic or elective purposes including dental impression acquisition that can be employed by consumers to obtain an impression of their teeth that can be used to form a custom-fit mouthguard.

### FIELD

The present technology relates to a kit for acquisition of human anatomy and a relationship of structures to each other for therapeutic or elective purposes including dental impression acquisition. More specifically, the technology relates to a kit that can be employed by consumers to obtain an impression of their teeth that can be used to form a custom-fit mouthguard.

### BACKGROUND

Mouthguards are dental devices that cover the wearer's teeth and protect the wearer's teeth, tongue, gums, and cheeks from trauma. Mouthguards are employed in various contexts. For example, mouthguards can be worn to protect the wearer's teeth during athletic activities. Mouthguards can also be worn at night to avoid teeth grinding or to address sleep apnea. Mouthguards can also be employed to treat temporomandibular disorders.

Mouthguards can be formed in different manners. Stock mouthguards are formed in a one-size-fits-all configuration and come preformed and ready to wear. Such stock mouthguards are not specifically designed for the wearer's teeth, and therefore rarely fit well or provide optimal performance. Their bulkiness and poor fit can make breathing difficult. Stock mouthguards offer the least protection of all mouthguard types. Other adjustable over-the-counter mouthguards have been developed having sliding pieces that rest between your teeth while you sleep. However, such mouthguards are not personalized and only provide limited adjustment for fit and performance. Further, they require additional elements to provide adjustability that can decrease comfort.

Boil-and-bite mouth protectors are formed from a thermoplastic material that the user can shape at home to fit their teeth. The mouthguard is softened by placing it in hot (not boiling) water. The softened mouthguard is then placed into the wearer's mouth and pressed onto the molars and front teeth. When the mouthguard is in the right position, the wearer bites down for about 20 seconds. The device is removed and placed under cool water to form the impression. While such mouthguards can be formed at home, they utilize thick materials that can be uncomfortable. Further, the accuracy of the mold is limited.

### BRIEF SUMMARY

The inventors of the present disclosure recognize that there are problems and limitations with conventional devices. For example, while custom fitted mouthguards fitted by a dentist provide the highest quality, fit, comfort, and performance with respect to protecting the wearer's teeth and mouth and the dentist forms a mold (impression) of the wearer's teeth, custom fitted mouth guards are higher cost and require a dental appointment. Over-the-counter mouthguards are available but are not personalized and only provide limited adjustments for fit and performance, and any adjustability features can decrease user comfort.

It is therefore a principal object and advantage of embodiments of the present technology to overcome these and other deficiencies in the art.

### BRIEF DESCRIPTION OF THE DRAWINGS

The present disclosure will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments.

Reference is now made briefly to the accompanying drawings, in which:

FIG. 1 is an image of an exemplary mouthguards, according to an embodiment of the present disclosure.

FIG. 2 is an image of spacer, according to an embodiment of the present disclosure.

FIG. 3 is an image of a dental acquisition kit, according to an embodiment of the present disclosure.

FIG. 4 is an image of a mouthguard, according to an embodiment of the present disclosure.

FIG. 5 is an image of a mouthguard in use, according to an embodiment of the present disclosure.

FIG. 6 is an image of mouthguards, according to an embodiment of the present disclosure.

### DETAILED DESCRIPTION

Embodiments of the present technology relate to a kit 10 for acquisition of human anatomy and relationship of structures to each other for therapeutic or elective purposes. For example, embodiments of the present technology relate to a dental acquisition kit 10 is intended to specifically reproduce dental oral structures including teeth, palate, maxillary and mandibular bone and gums, and the reproduction of the relationship between the upper and lower teeth as they occlude in a habitual bite of an individual and an altered bite. This can be called a therapeutic bite created through instructions of a pre-recorded video, scientific measurement such as the Shimbashi measurement, and/or created with the guidance of a dental provider via telemedicine or in person appointment. The dental acquisition kit 10 can also be intended for direct consumer communication of a patient's personal acquisition of their dental oral structures and the relationship of those structures to each other with a dental lab for the purpose of fabrication of oral appliances. The habitual bite of an individual/patient could be causing symptoms and alteration of the habitual bite with a therapeutic bite may be intended/suggested for treatment or in a design for other oral appliances, such as sports mouth guards to prevent injury. The habitual bite, as well as the therapeutic bite, translate the relationship of the mandible bone via the bite in a three-dimensional position in relationship to the maxillary bone and the relationship of the mandible to the skull. Studies have shown that the position of the mandible in relationship to the skull can alter the position of the head over the spine. This is significant, as the returned mandibular position results in a head posture in relationship to the spine for example, the head forward posture, which is associated undesirable symptoms such as neck pain, cervical pain, cervical disc compression, vertigo, and sleep apnea.

In accordance with an embodiment, the present technology relates to a kit 10 for dental impression acquisition. More specifically, the technology relates to a kit 10 that can be employed by consumers to obtain an impression of their teeth that can be used to fabricate a mouthguard 20. The technology may be employed to develop personalized mouthguards for sports, night wear, temporomandibular disorders, and sleep apnea. The kit 10 can be sold directly to consumers, for example as an over-the-counter product. The kit 10 allows consumers to take a dental impression and send the impressions directly to a lab to make a mouthguard for sports, a night guard, TMD relief, or sleep apnea, by way of example only. In one example, the kit 10 can be used during a telemedicine appointment to guide to user to take the dental impression. In other examples, the kit includes instructions that allow the consumer to take the dental impression without professional guidance or intervention.

The kit 10 is designed to acquire upper and lower teeth impressions of the user, and also provides materials to acquire a neuromuscular myobite measurement, which can be used to adjust the mouthguard to improve strength, flexibility, and balance during wear. Studies have shown that neuromuscular alignment of the mandible in relationship to the skull improves the head forward posture which causes conditions, such as sleep apnea, vertigo, balance issues, gait problems, ringing in the ears, neck, pain, shoulder pain, as well as other secondary pain from imbalance of the muscles, starting from head down the body.

An exemplary kit 10 for dental impression acquisition includes six cups of putty for forming the teeth impressions, including three cups of catalyst (which can include, for example, polyvinyl-siloxane, or any similar compositions (as should be understood and appreciated by those of ordinary skill in the art after a review of this disclosure), with, but not necessarily required, a 2 minute quick set, having a medium viscosity, low brittleness, and high flexibility), and three cups of putty base (as should be understood and appreciated by those of ordinary skill in the art after a review of this disclosure). Notably, mixing as little as three separate tablespoon scoops of catalyst and three separate tablespoons of base putty together induces a chemical reaction to harden the composition and a mold of structures is captured (yield preferred properties of the mixture), but the larger amount is provided in the kit 10 as may be needed. The kit can also include upper and lower trays (e.g., plastic, disposable, and/or perforated) for loading the putty and serving as a vehicle to contain the putty to 1) obtain impressions of upper teeth with the upper tray and lower teeth with the lower tray, or 2) be used as the frame structure of the final or temporary oral appliance device (mouthguard, sleep apnea oral device, night guard, temporomandibular repositioning device also called a TMD orthotic) with the hardened putty acting as the retention inside the trays, in the areas directly around the teeth, and the putty which extrudes out of the perforated exit holes 22 outside of the top of the tray (where typically the opposing teeth would connect against the teeth that are being impressed), this extra material that extrudes from the perforations to form a platform for the bite and impression or indents of the opposing teeth. These indents create a repetitive new landing area for the opposing teeth in the altered new improved bite position which has proven to improve neuromuscular symptoms, and in athletics shows to improve balance flexibility, power, and strength, increased breathing, and endurance. The kit can further include a spacer, ruler, and a Shimbashi chart for acquiring the neuromuscular myobite measurements, as described in further detail below.

Shimbashi standardized measurements can be based on a starting point of the width of the central incisor which provides average length of central incisor on average population and a healthy vertical height of dimension of occlusion in centric occlusion between the cervical fascial of upper right, central and seven locations of lower, right central tooth. See examples in the chart below:
1. 8 mm width of central (WOC)=10.5 mm length of central (LOC)=17 mm vertical dimension of occlusion (VDO)
2. 8.5 mm (WOC)=11 mm length (LOC)=17.75 mm VDO
3. 9. mm (WOC)=11 mm length (LOC)=18.5 mm VDO
4. 9.5 mm (WOC)=12.25 mm length (LOC)=20 mm VDO
5. 10 mm (WOC)=13 mm length (LOC)=21 mm VDO
6. _mm=_mm length=therapeutic temporary_mm VDO

The Shimbashi number was established to indicate an ideal vertical dimension of occlusion relationship between the upper and lower jaws. When there is an optimal vertical position of the lower jaw relative to the upper jaw, the muscles which are connected to them will also be at their optimal length and the muscles fibers can exhibit the optimal function. Although particular materials and elements of the exemplary kit 10 have been described, it is to be understood that other types and/or numbers of materials could be included in the kit for forming the dental impressions. For example, the kit 10 may include various spacers 24 of different sizes. They can be fabricated out of plastic or polyvinylsiloxane, and be in various lengths (i.e., 1 mm, 2 mm, 3 mm tall), or all could be 1 mm and clip/stack on each other. They can be placed on the top edge of the lower front two teeth and cover the midline contacts of the two front teeth. The shape can preferably be round so that it creates the smallest pinpoint contact during closure with the opposing teeth and prevent deflection of the jaw, as explained in further detail below. When the patient perceives the hard stop of the spacer 24, they will stop and hold the bite in that position until the putty hardens and sets. The spacer 24 the either becomes incorporated in the device, remains on the teeth and is removed from the teeth after impression is completed, or is removed from inside if the impression mold. This depends on what material the spacer 24 is made of, if the kit 10 is used for impressions and sending them to a dental lab for fabrication of the oral appliance of choice for the condition of the patient and/or healthcare provider is trying to treat, or for a final/temporary device the patient will wear. The role of the spacer 24 is to add vertical distance to the subject patient's closed vertical dimension measured from cervical buccal of the upper central tooth and cervical buccal of the lower central tooth located vertically below in the shortest distance. By adding height to the lower tooth with the spacer 24, the upper teeth connect against a hard stop of a specific vertical height and adds a vertical height to the preoperative vertical length between the cervical of upper central and cervical of the lower central. This is because the upper tooth contacts the lower tooth that is “taller”. This height is the lacking length to obtain the healthy Shimbashi measurement. For example, if the height vertical dime in centric occlusion between upper and lower teeth is 15, by adding length to the lower anterior of 2 mm in the vertical dimension should result as 17 mm by adding a 2 mm spacer on to the lower teeth is to perform the method of obtaining the neuromuscular myobite, as described in further detail below.

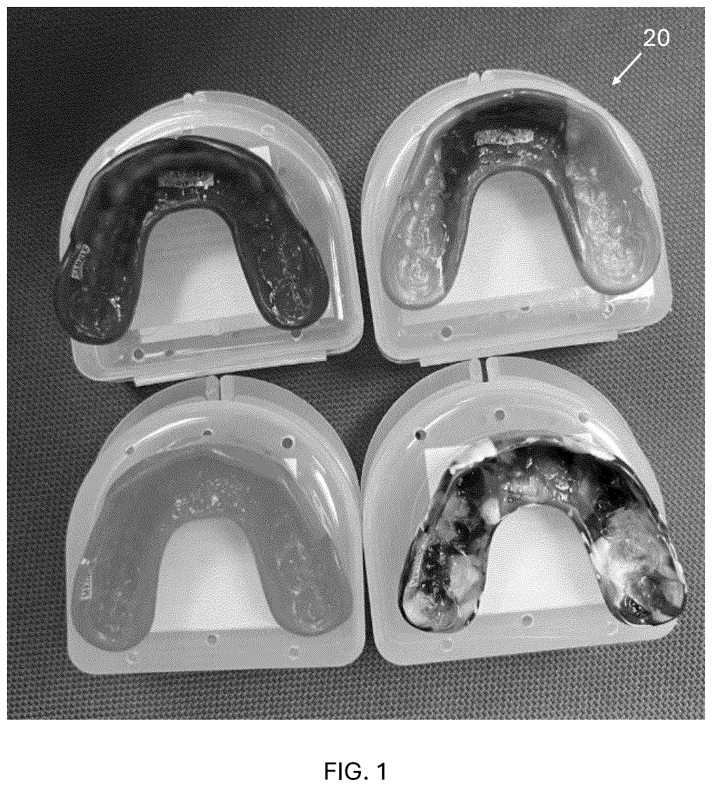

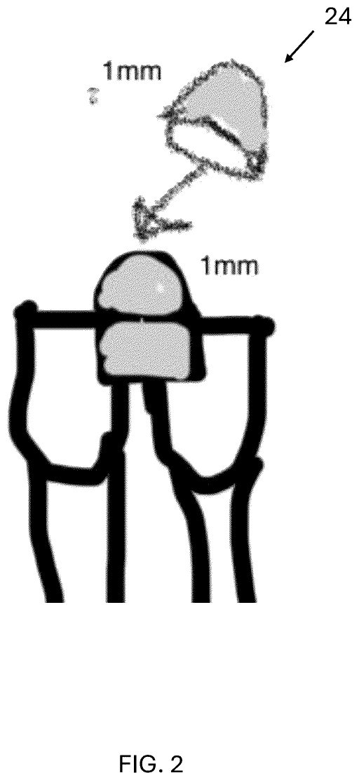

As shown in FIG. 2, a proposed spacer 24 design with a dome-shaped top and a clip to fit onto the edges of lower teeth. Addition of height could be by same height dome spacers 24, or the height can be preset, and the dome can be the exact height needed to compensate, for example, in FIG. 1 where 2 mm is needed, a 2 mm spacer dome can be placed. This does not exclude other designs of spacers, such as flat pieces that stack on top of and are configured to snap into (friction fit) each other like Lego® pieces, for example.

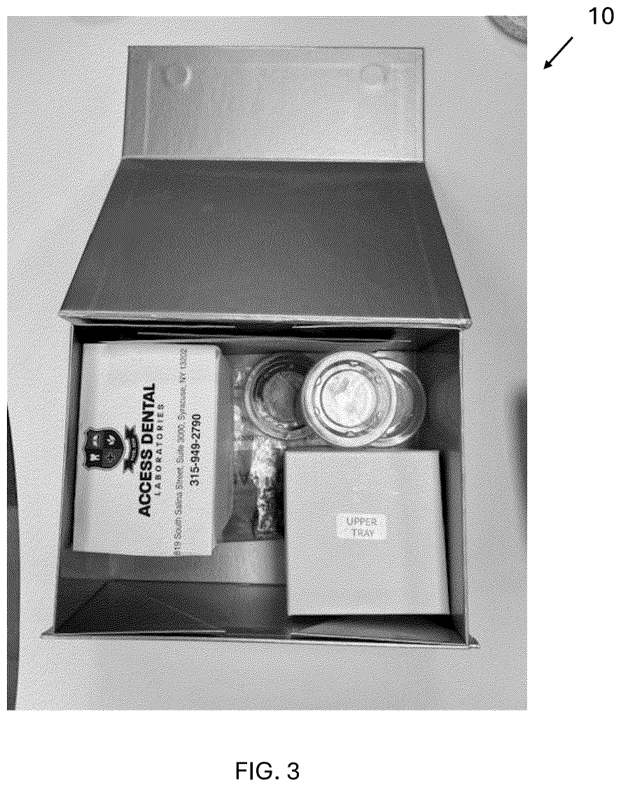

The kit 10 in one example also includes materials for the user to return the materials to a lab for fabricating the mouthguard. The materials can include a biohazard zip lock bag for inserting the trays after obtaining the impressions, and a return label and box. The kit 10 may also include in some examples a shipping return label to return the mouthguard from the lab to patient after fabrication. The kit 10 can also include other materials such as a registration form or one or more instruction documents to guide the user to form the dental impressions and to obtain the neuromuscular myobite in accordance with aspects of the present technology. An exemplary kit is illustrated in FIG. 3.

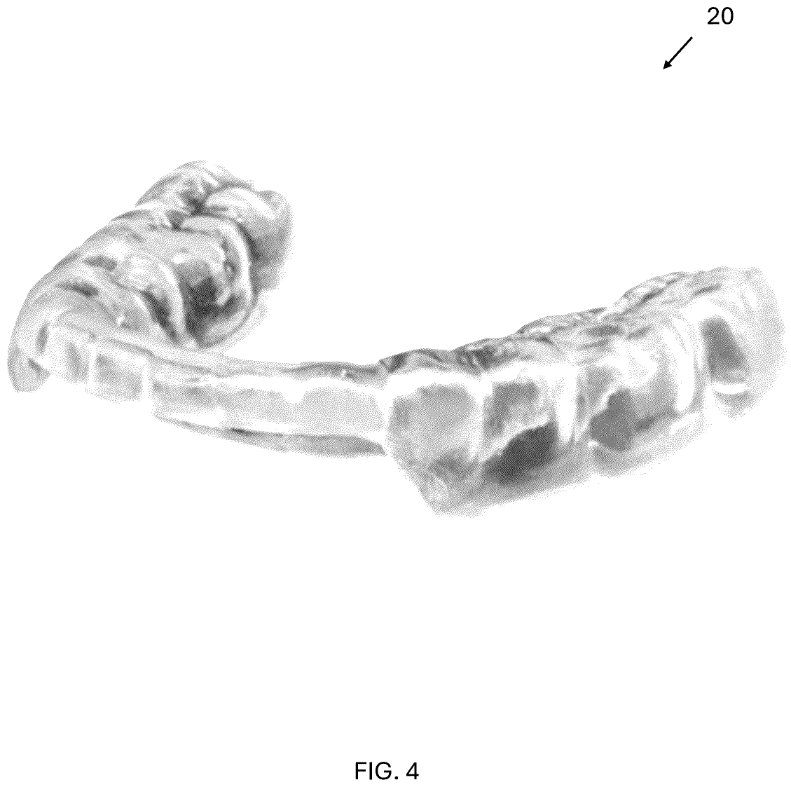

FIG. 4 illustrates the final product of a mouthguard 20 for use in treating TMD, by way of example only.

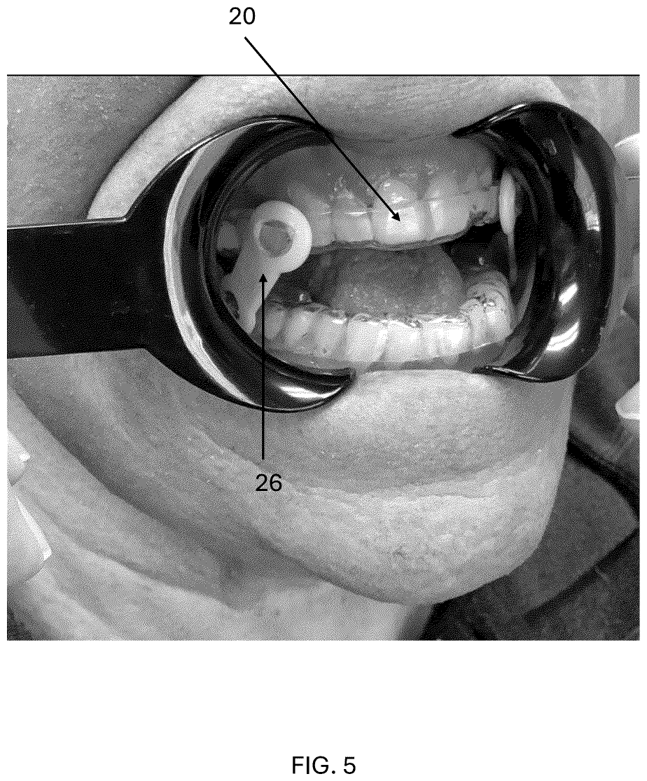

FIG. 5 illustrates the final product of a mouthguard 20 for use in treating sleep apnea, by way of example only. As shown, an appliance 26 can be connected to a mouthguard in order to keep a patient's jaw in an open position.

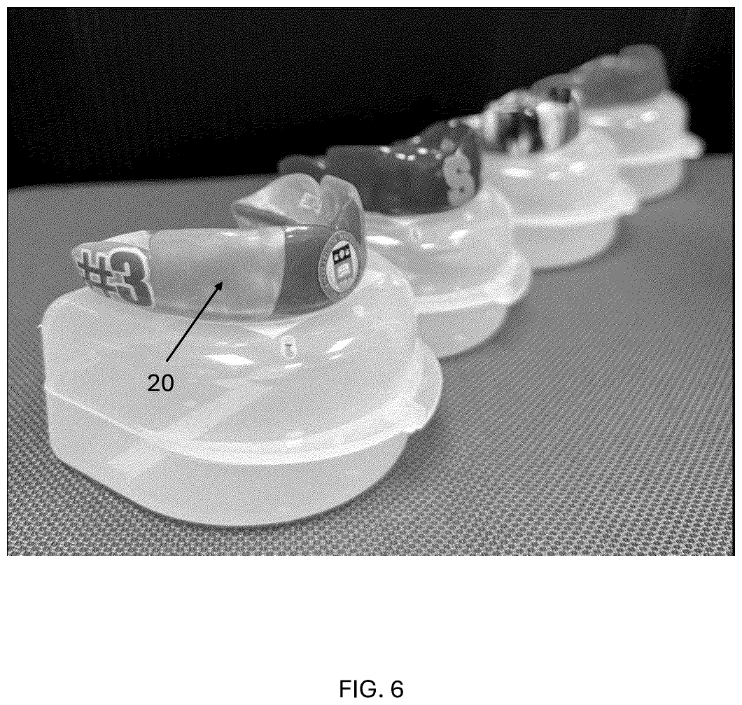

FIG. 6 illustrates the final product of a mouthguard 20 for use in sports, by way of example only.

An exemplary set of instructions for forming a dental impression using the kit of the present technology will now be described.

In a first step, the lower impression is formed. The user prepares one cup of blue putty and one cup of white putty and the lower tray. The blue and white putty are mixed together. Once the mixed putty begins to harden (within about 30 seconds), the user places the putty around the lower tray and flattens the putty down. The lower tray with putty mixture is inserted into the mouth and the putty is pressed into the user's teeth. The user should set the front teeth first and then work toward the back teeth to set the user's teeth into the putty. The user then holds the bite for approximately 2 minutes. The user then removes the lower tray from the mouth and places into the biohazard bag for shipment.

In a second step, the upper impression is formed. The user prepares one cup of blue putty and one cup of white putty and the upper tray. The blue and white putty are mixed together. Once the mixed putty begins to harden (within about 30 seconds), the user places the putty around the upper tray and flattens the putty down. The upper tray with putty mixture is inserted into mouth and the putty is pressed into the user's teeth. The user should set the front teeth first and then work toward the back teeth to set the user's teeth into the putty. The user then holds the bite for approximately 2 minutes. The user then removes the upper tray from the mouth and places into the biohazard bag for shipment.

In one example, the user can fill out registration form, for example by scanning a QR code on the registration form. The biohazard bag can then be placed in the return box and returned using the shipping label enclosed in the kit. Various packaging and shipping methods may be employed. In another example, the user can also generate a neuromuscular myobite in accordance with the exemplary instructions discussed below.

An exemplary set of instructions for performing the neuromuscular myobite will now be described. The instructions provide a standardized method of teeth and bite and vertical dimension acquisition for optimal neuromuscular position of the upper and lower jaw in fabrication of a sports mouthguard, TMD, orthotic, appliance, night guard for grinding, and sleep apnea oral appliance. The instructions can be included in the kit 10 along with the other materials to perform the neuromuscular myobite. In another example, the neuromuscular myobite can be performed using doctor guidance to obtain optimal performance, although the method may be performed solely based on the instructions included in the kit.

First, the user places a chair against wall on flat surface and prepares one cup each of the blue and white putty (without mixing). The user then sits in the chair with feet flat on the ground and no shoes on, making sure the head is straight and the user is looking forward. The user's eyes need to be horizontal and looking forward. The user should sit up straight and keep the chest out. The user opens and closes the jaw without having the teeth touch. The user wiggles the jaw side to side, and opening and closing to loosen the jaw. The user massages the sides of the jaw and temples.

The goal is to obtain a bite where the first pinpoint contact between the front teeth occurs. The user can measure the width of their upper central in the widest part of the upper right front. They will circle that number in the Shimbashi chart provided, as explained above.

The user will bite together and using a ruler included in the kit, measure the distance between the “neck” cervical buccal of the of upper right central and lower cervical buccal of lower central, in the closed position called centric occlusion. They will write this length down in the table provided. The user will locate the ideal vertical distance that corresponds to their eight central measurements they circled on the chart. The user will subtract the obtained vertical distance with the ruler that they wrote down from the ideal vertical that corresponds to the width of the central tooth they circled. The resulting difference is the height of the spacer needed to be added to the lower front teeth in the area of the midline between the two lower front teeth. Once the spacer is placed on the lower front tooth, the user will mix the remaining tablespoon of catalyst and tablespoon of base. While they are mixing the material, it is important for the user to “deprogram” their bite and relax the masseter muscles by opening and closing their mouth without touching their teeth together for more than 30 times, for example. This exercise can be crucial in obtaining a neutral deprogrammed bite. The use will open and close (almost biting but still no contact between the teeth) also “wiggle” the lower jaw from right to left as they open and close. This breaks up muscle spasms and allows for isotonic muscle fiber position.

Finally, the user will take a bite by placing a rolled out “hotdog” shaped putty on top of lower teeth, including over the spacer. They will open wide one last time, “wiggle” side to side, then start closing the mandible vertically until they perceive the first front pinpoint contact between the front upper and lower teeth occurs and hold that bite until the putty sets. This area is marked on a sheet/chart included in the kit. While keeping the jaw open and loose, the user mixes the blue and white putty together and rolls the putty mixture into a hotdog shaped form. The putty is placed on the user's bottom teeth without pressing down on the putty. The user opens the jaw very wide, then starts to close into the putty mixture vertically without swinging the jaw until they “hit” and perceive that there is a first point of contact. The user will hold the bite until putty hardens.

After the user finds the point of interference, the spacer 24 is placed on top of that tooth, and the bite is completed and held for approximately two minutes. The kit 10 advantageously includes the spacer for obtaining the myobite. The spacer 24 is placed between the teeth and provides an optimal overjet and overbite between the top and bottom teeth, which results in the standardized Shimbashi vertical measurement between the cervical of the upper front tooth and the cervical of the lower front tooth.

The Shimbashi measurement has been studied and provides a guideline for how much a jaw must be opened to provide an ideal neuromuscular position. This measurement is standardized based on the width of the central incisor and provides the ideal size, length of the central incisor, and also the ideal vertical measurement between the upper and lower tooth in healthy individuals. In the event that this vertical dimension is decreased below that average vertical dimension, studies have shown that patients show pathology and symptoms. In some examples, the kit includes a Shimbashi chart to allow the user to determine an appropriate Shimbashi number for reference in performing the disclosed method using the kit.

When acquiring the myobite, it is important for the user to acquire the vertical dimension that displays in average healthy patients based on the width of the central incisor. To accomplish this, the spacer 24 is placed between the lower front teeth to provide a standardized measurement in millimeters to compensate for the loss of vertical dimension. For example, if the patient measures 14 mm, and an idea width for that central incisor is 17 mm, the spacer 24 will be 3 mm tall and add three millimeters to the lower central incisor. In this manner, the spacer 24 prevents over closure during the acquisition of the bite. The spacer 24 will come in various measurements to provide the proper correction.

The patient will measure the distance when they close their teeth between the neck of the upper tooth and the neck of the lower tooth right below it, and note the vertical starting dimension with the ruler provided. The patient then will measure the width of the front upper tooth and note the width. The patient will be provided with a Shimbashi chart and look up the ideal vertical dimension, matched up to the width of the central incisor front tooth. Next the patient will subtract their current vertical dimension from the ideal vertical dimension on the chart. The result in millimeters provides the proper size of the spacer required.

As set forth above, the kit 10 may include various size spacers 24 to accommodate different measurements. The patient uses the correct spacer 24 based on the size in millimeters that matches the result needed to open them up to the ideal, healthy, vertical dimension. For example, when the patient measures 14 mm and the chart suggested 17 mm. Therefore, there is a deficiency of 3 mm. The patient will select a 3 mm spacer 24 to place on his lower front tooth before acquiring the bite.

Once the user completes the bite, the putty can be placed in the biohazard bag and returned to the lab for fabrication using the packaging and label included in the kit 10. The lab can then fabricate the mouthguard to the user's customization. Exemplary fabrication techniques can include, but are not limited to, 3D printing of a custom-fit mouthguard using the bite acquired as described above.

The kit 10 of the present technology can be used to create mouthguards 20 for various exemplary uses, including those listed below.

In one example, the kit 10 can be used to form a mouthguard 20 for sports applications. The fabricated mouthguard 20 can be used to protect the user's dentition during agility and high-level performance for all sports. Additionally, the mouthguard 20 can be fabricated using the neuromuscular myobite to apply the science of neuromuscular dentistry and neuromuscular myobite relationship of the mandible position. This relationship allows for optimizing the position of the head over the spine and provides the athlete the optimum body and core strength, endurance, and persistence during competitive sports where time, measure, and weight will allow the athlete to perform at their best. In one example, the mouthguard can also be customized with decals that within the material that incorporate sayings, logos, phrases, pictures, or other designs.

In another example, the kit 10 can be used to form a mouthguard 20 to be worn by the user at night to avoid teeth grinding. Over 75% of the population suffers from clenching or grinding teeth, and sleep disturbance leading to tooth destruction and restless nights. Currently available over the counter appliances available to consumers have been in soft material guards which encourage the clenching and grinding habit and do not work. Other hard material guards are bulky and require frequent dental visits. The present technology provides a hard mouthguard 20 that can prevent grinding while being customized for the user. The device of the present technology advantageously targets and eases more symptoms associated with the bite, and other problems relating to jaw positioning and sleep disturbance. The present technology is also less expensive, and the impressions can be obtained at home using the kit. Further, along with grinding there are other problems that can be treated at the same time which can be incorporated into the mouthguard (such as TMD).

In a further example, the kit 10 of the present technology can be used to form a mouthguard 20 for treating TMD, where structures of the head are misaligned to a degree that causes the muscular to throw the body out of alignment from the head down. The present technology provides a kit for making a mouthguard 20 that aligns the user's neuromuscular system from the head down. The secret is to put the head over the spine to align your entire body which gives the user the upmost core strength with a straight line alignment versus an incontiguous line.

In yet a further example, the kit 10 of the present technology can be used to form a mouthguard 20 for treating sleep apnea. The secret of advancing the jaw to improve a collapsed and blocked airway has been studied thoroughly and fabricated for years in the dentist office as an alternative to a C-Pap intolerant patients. The present technology provides a cost-effective solution that can be employed in a convenient doctor guided process in the convenience of the user's home. As shown in FIG. 5 and explained above, an appliance 26 can connect to the mouthguard 20 in order to keep a patient's jaw advanced.

Accordingly, the present disclosure provides a number of advantages including providing a dental impression acquisition kit 10 and methods of use therefore that allows a user to form impressions necessary to create a customized mouthguard in the home. The technology also includes materials necessary to generate a neuromuscular myobite that can be used to customize the mouthguard to provide neuromuscular benefits to the wearer.

Aspects of the present invention and certain features, advantages, and details thereof, are explained more fully below with reference to the non-limiting examples illustrated in the accompanying drawings. Descriptions of well-known structures are omitted so as not to unnecessarily obscure the invention in detail. It should be understood, however, that the detailed description and the specific non-limiting examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure. The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as, “has” and “having”), “include” (and any form of include, such as “includes” and “including”), and “contain” (any form of contain, such as “contains” and “containing”) are open-ended linking verbs. As a result, a method or device that “comprises”, “has”, “includes” or “contains” one or more steps or elements. Likewise, a step of method or an element of a device that “comprises”, “has”, “includes” or “contains” one or more features possesses those one or more features but is not limited to possessing only those one or more features. Furthermore, a device or structure that is configured in a certain way is configured in at least that way, but may also be configured in ways that are not listed.

The corresponding structures, materials, acts and equivalents of all means or step plus function elements in the claims below, if any, are intended to include any structure, material or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the present invention for various embodiments with various modifications as are suited to the particular use contemplated.

What is claimed is:

1. A kit for obtaining a dental impression, the kit comprising:
a first material and a second material configured to be mixed into a third material for receiving a dental impression therein;
an upper and lower tray for forming an impression of both an upper and lower set of teeth, respectively; and
a set of instructions to guide a user to form the dental impression comprising:
mixing the first and material and the second material to form the third material;
applying the third material to both the upper and lower trays;
inserting the upper tray and lower into the user's mouth to form the dental impression of the user's upper and lower teeth, respectively.

2. The kit of claim 1, wherein the first material and second materials are putty.

3. The kit of claim 1, wherein the instructions further comprise:
allowing the third material to harden for at least thirty seconds before performing the applying step.

4. The kit of claim 1, wherein the instructions for the applying include applying the upper and lower tray sequentially.

5. The kit of claim 1 further comprising:
a biohazard bag for collecting the formed dental impressions; and
packing materials for sending the biohazard bag to a lab for fabrication of a mouthguard using the formed dental impressions.

6. The kit of claim 1, further comprising materials for generating a neuromuscular myobite comprising:
a spacer device; and
a chart for the user to determine a Shimbashi number, wherein the spacer is employed to correct the user's mandible position to match the Shimbashi number from the chart, wherein the instructions further comprise:
placing the third material on the user's bottom teeth;
identifying a first point of contact between the user's upper and lower teeth during a bite performed by the user;
placing the spacer on the bottom tooth at the first point of contact; and
completing a bite into the third material with the spacer in place to form the neuromuscular myobite.

7. The kit of claim 6 further comprising:
a biohazard bag for collecting the formed neuromuscular myobite; and
packing materials for sending the biohazard bag to a lab for fabrication of a mouthguard using the formed dental impressions and the formed neuromuscular myobite.
